(19) United States
(12) Patent Application Publication
(10) Pub. No.: US 2005/0137255 A1
(43) Pub. Date: Jun. 23, 2005

(54) Crystalline escitalopram hydrobromide and methods for preparing the same

(75) Inventors: Hans Petersen, Vanlose (DK); Peter Ellegaard, Jystrup (DK); Lawrence Martel, Manchester, NH (US); Robert Dancer, Hvidovre (DK)

Correspondence Address: DARBY & DARBY P.C., P. O. BOX 5257, NEW YORK, NY, 10150-5257, US

(73) Assignee: H. Lundbeck A/S, Copenhagen-Valby (DK)

(21) Appl. No.: 11/025,532

(22) Filed: Dec. 29, 2004

Related U.S. Application Data
(63) Continuation-in-part of application No. 10/746,913, filed on Dec. 23, 2003.
(60) Provisional application No. 60/584,414, filed on Jun. 30, 2004.

(30) Foreign Application Priority Data
Dec. 23, 2002 (DK) PA 2002 02005

(51) Int. Cl. A61K 31/343; C07D 307/78

(52) U.S. Cl. 514/469; 549/467

(57) ABSTRACT

The present invention provides crystalline escitalopram hydrobromide ((S)-1-[3-(dimethylamino)propyl]-1-(4-fluorophenyl)-1,3-dihydro-5-isobenzo-furan carbonitrile hydrobromide), and a novel crystalline form of escitalopram hydrobromide referred to herein as Form I. Form I is stable, water soluble, and not hygroscopic at a relative humidity less than 70%.

This application claims the benefit of U.S. Provisional Application No. 60/584,414, filed Jun. 30, 2004, and is a continuation-in-part of U.S. patent application Ser. No. 10/746,913, filed Dec. 23, 2003, which claims the benefit of Danish Patent Application No. PA 2002 02005, filed Dec. 23, 2002, all of which are hereby incorporated by reference in their entireties.

### FIELD OF INVENTION

The present invention is directed to a crystalline escitalpram hydrobromide ((S)-1-[3-(dimethylamino)propyl]-1-(4-fluorophenyl)-1,3-dihydro-5-isobenzofuran carbonitrile hydrobromide), pharmaceutical compositions containing the same, and methods of preparing the same.

### BACKGROUND OF THE INVENTION

Citalopram is a well known antidepressant drug that has been widely sold for many years and has the following structure 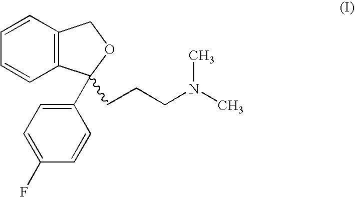
It is a selective, centrally acting serotonin (5-hydroxytryptamine; 5-HT) reuptake inhibitor accordingly having antidepressant activities. Citalopram was first disclosed in DE 2,657,013 corresponding to U.S. Pat. No. 4,136,193.

The S-enantiomer of citalopram (escitalopram) has the formula 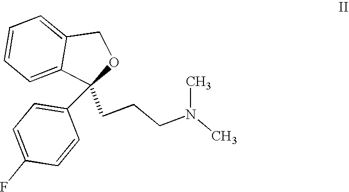
and was described along with its antidepressant effect in U.S. Pat. No. 4,943,590. EP patent application 1.200.081 describes the use of escitalopram for the treatment of neurotic disorders and WO02/087566 describes the use of escitalopram for the treatment of depressed patients who have failed to respond to conventional selective seritonin reuptake inhibitors (SSRI's) in addition to other disorders.

Methods for the preparation of escitalopram are disclosed in U.S. Pat. No. 4,943,590. This patent also describes the free base of escitalopram as existing as an oil as well as the oxalic, pamoic and L-(+)-tartaric acid addition salts of escitalopram.

In the search for salts of escitalopram suitable for pharmaceutical composition more than 30 organic and inorganic acids were investigated under different conditions. These acids gave either oils or amorphous solids having moderate to high hygroscopic properties. The non-hydroscopic crystalline solids were formed from non-carboxylic organic acids, indeed most of the addition salts formed with monocarboxylic organic acids. Di- and triphasic organic acids gave amorphous solids as did the salt formed with L-tartaric acid.

Thus, very few crystalline stable, non-hydroscopic salts of escitalopram are known.

### SUMMARY OF THE INVENTION

The present invention provides crystalline escitalopram hydrobromide ((S)-1-[3-(dimethylamino)propyl]-1-(4-fluorophenyl)-1,3-dihydro-5-isobenzofuran carbonitrile hydrobromide), and a novel crystalline form of escitalopram hydrobromide referred to herein as Form I. Form I is stable, water soluble, and not hygroscopic at a relative humidity less than 70%.

Another embodiment is a pharmaceutical composition comprising crystalline escitalopram hydrobromide (such as Form I escitalopram hydrobromide) and, optionally, a pharmaceutically acceptable excipient. According to one embodiment, the pharmaceutical composition comprises a therapeutically effective amount of crystalline escitalopram hydrobromide or crystalline Form I of escitalopram hydrobromide. For example, the pharmaceutical composition can comprise an amount of crystalline escitalopram hydrobromide or crystalline Form I escitalopram hydrobromide effective to treat escitalopram-treatable disorders in a subject, such as a mammal (e.g. human). According to one preferred embodiment, the pharmaceutical composition comprises at least about 20, 30, 40, 50, 60, 70, 80, 90, 95, 96, 97, 98, 99, 99.1, 99.2, 99.3, 99.4, 99.5, 99.6, 99.7, 99.8, or 99.9% by weight of crystalline escitalopram hydrobromide or crystalline Form I of escitalopram hydrobromide, based upon 100% total weight of escitalopram hydrobromide in the pharmaceutical composition. According to another preferred embodiment, the pharmaceutical composition comprises at least about 20, 30, 40, 50, 60, 70, 80, 90, 95, 96, 97, 98, 99, 99.1, 99.2, 99.3, 99.4, 99.5, 99.6, 99.7, 99.8, or 99.9% by weight of Form I of escitalopram hydrobromide, based upon 100% total weight of crystalline escitalopram hydrobromide in the pharmaceutical composition. The pharmaceutical composition may be in the form of a unit dosage form, such as a tablet or capsule. According to one embodiment, the unit dosage form contains from about 2.5 to 20 mg of escitalopram hydrobromide (such as 5, 7.5, or 10 mg) (calculated based on the weight of escitalopram free base).

Yet another embodiment is a method of treating a subject (such as a mammal (e.g., human)) having an escitalopram-treatable disorder comprising administering a therapeutically effective amount of a pharmaceutical composition comprising crystalline escitalopram hydrobromide or crystalline Form I of escitalopram hydrobromide.

Yet another embodiment is a method for preparing crystalline escitalopram hydrobromide comprising the steps of:
(a) forming an anhydrous solution of escitalopram hydrobromide and at least one organic solvent (e.g., iso-propanol); and
(b) precipitating crystalline escitalopram hydrobromide from the anhydrous solution.

Yet another embodiment is a method for preparing crystalline escitalopram hydrobromide comprising the steps of:
(a) dissolving escitalopram free base in iso-propanol;
(b) adding aqueous hydrobromic acid;
(c) drying the solution (such as by azeotropic distillation or adding a solid drying agent); and
(d) precipitating crystalline escitalopram hydrobromide from the solution.

### BRIEF DESCRIPTION OF THE DRAWINGS

FIG. 1 is a characteristic X-ray powder diffraction (XRPD) pattern for Form I of escitalopram hydrobromide.

FIG. 2 is a drawing derived from the crystal structure of Form I of escitalopram hydrobromide, that shows the conformation of the molecule in the structure.

FIG. 3 is a differential scanning calorimetry (DSC) thermogram of Form I of escitalopram hydrobromide.

FIG. 4 is a thermogravimetric analysis (TGA) thermogram of Form I of escitalopram hydrobromide.

FIG. 5 is a dynamic vapor sorption (DVS) plot of Form I of escitalopram hydrobromide.

### DETAILED DESCRIPTION OF THE INVENTION

The term “about” generally means within 10%, preferably within 5%, and more preferably within 1% of a given value or range. With regard to a given value or range in degrees 2θ from XRPD patterns, the term “about” generally means within 0.2° 2θ and preferably within 0.1°, 0.05°, or 0.01° 2θ of the given value or range. Alternatively, the term “about” means within an acceptable standard error of the mean, when considered by one of ordinary skill in the art.

The term “escitalopram hydrobromide” refers to (S)-1-[3-(dimethylamino)propyl]-1-(4-fluorophenyl)-1,3-dihydro-5-isobenzofuran carbonitrile hydrobromide.

A “pharmaceutically acceptable excipient” refers to an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient. Suitable pharmaceutically acceptable excipients include, but are not limited to, carriers, diluents, flavorants, sweeteners, preservatives, dyes, binders, suspending agents, dispersing agents, colorants, disintegrants, lubricants, plasticizers, edible oils, and any combination of any of the foregoing.

“Treating” or “treatment” of a state, disorder or condition includes:

(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,

(2) inhibiting the state, disorder or condition, i.e., arresting or reducing the development of the disease or at least one clinical or subclinical symptom thereof, or

(3) relieving the disease, i.e., causing regression of the state, disorder or condition or at least one of its clinical or subclinical symptoms. The benefit to a subject to be treated is either statically significant or at least perceptible to the patient or to the physician.

A “therapeutically effective amount” means the amount of escitalopram hydrobromide that, when administered to a mammal for treating a state, disorder or condition, is sufficient to effect such treatment. The “therapeutically effective amount” will vary depending on the state, disorder or condition and its severity and the age, weight, physical condition and responsiveness of the subject to be treated.

“Escitalopram-treatable disorders” include, but are not limited to, depression (e.g., major depression disorder and treatment of patients which failed to respond to initial treatment with conventional selective serotonin reuptake inhibitors (SSRIs)), neurotic disorders (including, but not limited to, panic attacks (including, but not limited to, panic attacks associated with panic disorder, specific phobias, social phobia and agoraphobia), post traumatic stress disorder, obsessive compulsive disorder, and anxiety states such as generalized anxiety disorder and social anxiety disorder), acute stress disorder, eating disorders (such as bulimia, anorexia and obesity), phobias, dysthymia, premenstrual syndrome, premenstrual dysphoric disorder, cognitive disorders, impulse control disorders, attention deficit hyperactivity disorder, and drug abuse. The term “escitalopram-treatable disorders” also includes disorders for which escitalopram is known to be an effective treatment, such as those described in International Publication Nos. WO 01/03694 and WO 02/087566, both of which are incorporated by reference.

### Form I of Escitalopram Hydrobromide

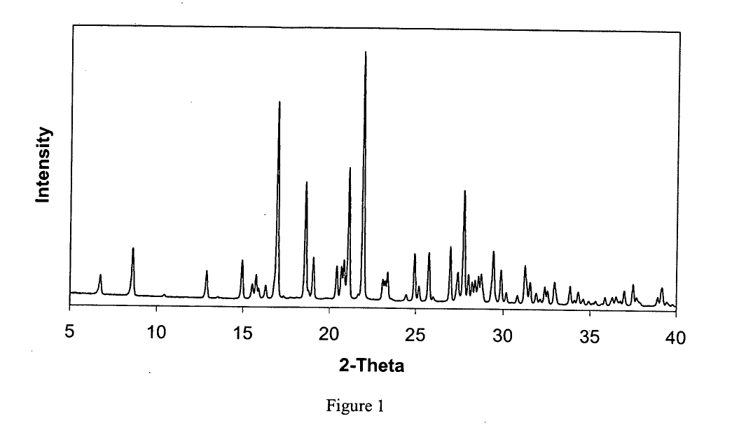

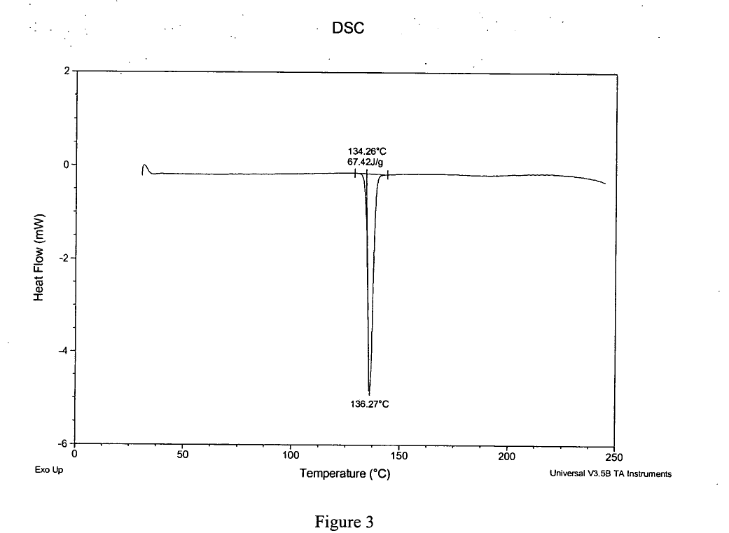

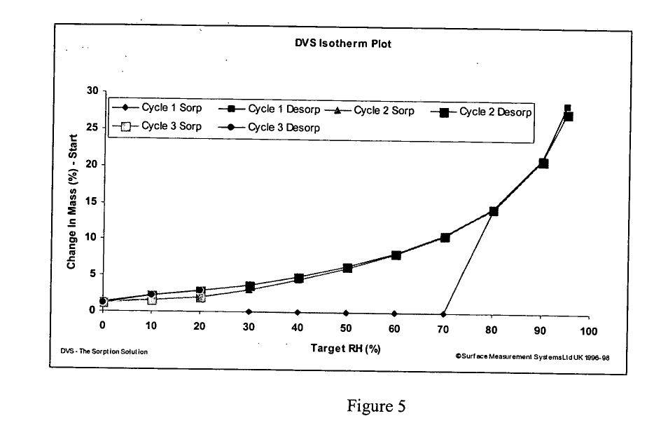

Form I has a distinct XRPD pattern as shown in FIG. 1. The term “Form I” as used herein refers to crystalline forms of escitalopram hydrobromide having this and substantially related XRPD patterns. Positions of some characteristic reflections in the XRPD pattern (using CuK_{α1} radiation) of Form I are provided in Table 1 below. The peak (expressed in degrees 2θ±0.10°) at 21.9 is characteristic of Form I. Other characteristic peaks (expressed in degrees 2θ±0.1°) include those at 16.95, 18.59, 21.1, and 27.76. Form I has a melting point onset as measured by differential scanning calorimetry at from about 131 to about 135° C. (see, e.g., FIG. 3). FIG. 5 shows the dynamic vapor sorption (DVS) curves for Form I. As shown by FIG. 5, Form I of escitalopram hydrobromide is non-hygroscopic at a relative humidity less than about 70%. At a relative humidity above 70%, the escitalopram hydrobromide absorbs water and turns into a sticky oil. Subsequent drying of the sticky oil does not return the escitalopram hydrobromide to a crystalline solid.

TABLE 1
Characteristic XRPD Peaks (expressed in degrees 2θ ± 0.1° 2θ)
and Intensities of Diffraction Lines for Form I
Degrees 2θ(±0.2° 2θ)

6.760
8.640
12.910
14.960
16.950
18.590
19.050
20.410
21.100
21.930
24.940
25.750
26.990
27.420
27.760
29.430
29.870

Form I exhibits a single crystal X-ray crystallographic analysis at 122±2 K with crystal parameters that are approximately equal to the following:

| Parameter | Form I |
|---|---|
| Space group | Orthorhombic P2,2,2, |
| Cell Dimensions | |
| a(Å) | 6.5456(8) Å |
| b(Å) | 11.0611(6) Å |
| c(Å) | 25.795(3) Å |
| Volume (Å³) | 1867.6(3) |
| Z (molecules/unit cell) | 4 |
| Density | 1.442 g/cm³ |

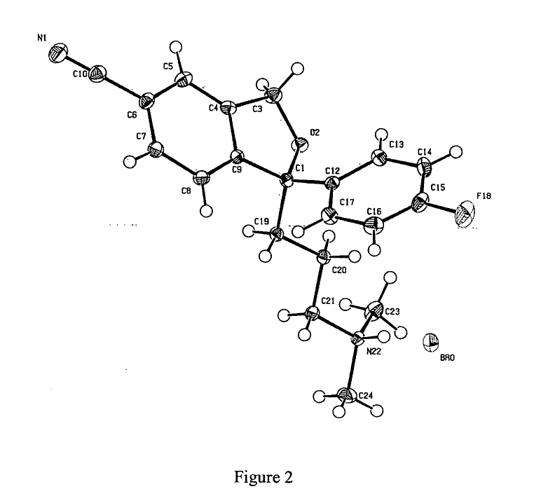

(the numbers in parenthesis are standard deviations on the last digit)
A drawing derived from the crystal structure of Form I of escitalopram hydrobromide, that shows the conformation of the molecule in the structure is shown in FIG. 2.

The atomic positions in Form I are provided in tables 2 and 3 below.

TABLE 2
Atomic Coordinates (non-H atoms)

| label | x | y | z |
|---|---|---|---|
| F(18) | 0.4146(2) | 0.29703(11) | 0.88097(5) |
| O(2) | 1.01516(18) | 0.73348(11) | 0.90561(4) |
| N(1) | 0.5995(3) | 1.24705(16) | 1.03267(6) |
| N(22) | 1.0109(2) | 0.61741(12) | 0.70490(5) |
| C(1) | 0.8097(3) | 0.74810(14) | 0.88553(6) |
| C(3) | 1.0350(3) | 0.79270(16) | 0.95471(7) |
| C(4) | 0.8639(2) | 0.88254(14) | 0.95558(6) |
| C(5) | 0.8340(3) | 0.98174(15) | 0.98712(6) |
| C(6) | 0.6599(3) | 1.05267(14) | 0.97856(6) |
| C(7) | 0.5192(3) | 1.02336(15) | 0.93982(6) |
| C(8) | 0.5526(2) | 0.92303(17) | 0.90804(6) |
| C(9) | 0.7266(2) | 0.85409(14) | 0.91616(6) |
| C(10) | 0.6271(3) | 1.16083(17) | 1.00909(7) |
| C(12) | 0.6915(3) | 0.62971(14) | 0.89181(6) |
| C(13) | 0.7886(3) | 0.52656(15) | 0.91024(6) |
| C(14) | 0.6941(3) | 0.41417(16) | 0.90792(6) |
| C(15) | 0.5023(3) | 0.40822(17) | 0.88644(7) |
| C(16) | 0.3959(3) | 0.50842(18) | 0.87030(7) |
| C(17) | 0.4914(3) | 0.62002(15) | 0.87308(7) |
| C(19) | 0.8277(3) | 0.77951(13) | 0.82781(6) |
| C(20) | 0.9313(3) | 0.68157(15) | 0.79576(7) |
| C(21) | 0.9153(3) | 0.71288(15) | 0.73841(6) |
| C(23) | 1.2319(3) | 0.59875(19) | 0.71576(7) |
| C(24) | 0.9768(4) | 0.64550(19) | 0.64904(7) |
| Br(0) | 0.32633(2) | 0.915787(14) | 0.772754(7) |

TABLE 3
Atomic Coordinates (H atoms)

| label | x | y | z |
|---|---|---|---|
| H(3A) | 1.161(4) | 0.8295(19) | 0.9562(8) |
| H(3B) | 1.029(3) | 0.7323(19) | 0.9830(7) |
| H(5) | 0.929(3) | 1.0060(18) | 1.0131(8) |
| H(7) | 0.410(3) | 1.074(2) | 0.9357(8) |
| H(8) | 0.462(3) | 0.9063(19) | 0.8806(8) |
| H(13) | 0.919(4) | 0.532(2) | 0.9239(8) |
| H(14) | 0.758(3) | 0.345(2) | 0.9189(8) |
| H(16) | 0.265(4) | 0.497(2) | 0.8551(10) |
| H(17) | 0.420(4) | 0.689(2) | 0.8621(9) |
| H(19A) | 0.695(3) | 0.7955(17) | 0.8151(7) |
| H(19B) | 0.908(3) | 0.8523(19) | 0.8248(8) |
| H(20A) | 0.870(3) | 0.6028(17) | 0.8011(7) |
| H(20B) | 1.077(4) | 0.681(2) | 0.8060(9) |
| H(21A) | 0.775(3) | 0.7196(19) | 0.7271(8) |
| H(21B) | 0.993(3) | 0.7871(16) | 0.7301(7) |
| H(23A) | 0.941(3) | 0.550(2) | 0.7124(8) |
| H(23B) | 1.290(4) | 0.545(2) | 0.6891(9) |
| H(23C) | 1.246(4) | 0.559(2) | 0.7496(10) |
| H(24A) | 1.305(4) | 0.672(2) | 0.7171(9) |
| H(24B) | 1.040(3) | 0.584(2) | 0.6279(8) |
| H(24C) | 1.040(4) | 0.717(2) | 0.6430(10) |

Crystalline escitalopram hydrobromide and crystalline Form I of escitalopram hydrobromide may be prepared by precipitating it from an anhydrous solution of escitalopram hydrobromide and at least one organic solvent.

Suitable organic solvents include, but are not limited to, iso-propanol, toluene, methyl t-butyl ether, a mixture of methyl t-butyl ether and isopropanol, tetrahydrofuran, butanone, n-butanol, iso-butanol, tert-butanol, a mixture of tert-butanol and isopropanol, 2-butanol, methyl iso-butyl ketone, 2-methyl-tetrahydrofuran, 1,4-dioxane, diethyl ether, ethyl acetate, acetone, and any combination of any of the foregoing. A preferred organic solvent is iso-propanol. Preferably, the organic solvent is one that does not readily pick up water (i.e., is not hygroscopic).

The anhydrous solution may be formed by introducing hydrobromide gas (e.g., by bubbling) into a solution of escitalopram free base and iso-propanol to form escitalopram hydrobromide. The solvent may be changed from iso-propanol to another organic solvent by concentrating the iso-propanol solution and dissolving the resulting escitalopram hydrobromide in at least one organic solvent (such as any of those mentioned above (e.g., acetone)) to form the anhydrous solution.

The anhydrous solution may also be formed by adding a solution of hydrobromide (e.g., 0.9-1.0 eq.) and iso-propanol to a solution of escitalopram free base (e.g., about 20% w/w) and iso-propanol. According to one embodiment, the addition is performed slowly such as by dropwise addition. The solvent may be changed from iso-propanol to another organic solvent (e.g., a 0.5 molar solution) by concentrating the iso-propanol solution and dissolving the resulting escitalopram hydrobromide in at least one organic solvent (such as any of those mentioned above (e.g., acetone)) to form the anhydrous solution.

Crystalline escitalopram hydrobromide and crystalline Form I of escitalopram hydrobromide may also be prepared by dissolving escitalopram free base in iso-propanol, adding aqueous hydrobromic acid (e.g., 0.9-1.0 eq.), and drying the solution to remove any water present. The drying can be performed by azeotropic distillation or repeated azeotropic distillation (e.g., with iso-propanol and toluene). The drying can also be performed by adding a solid drying (e.g., magnesium sulfate, molecular sieves) agent to the solution.

Escitalopram free base may be prepared by any method known in the art, such as those described in U.S. Pat. Nos. 4,593,590 and 6,566,540 and International Publication Nos. WO 03/000672, WO 03/006449, and WO 03/051861, all of which are hereby incorporated by reference.

### EXAMPLES

The following examples are illustrative and are not meant to limit the scope of the claimed invention.

### Example 1

(A) A 250 ml round bottom flask was charged with 5.7 g escitalopram free base and 120 ml isopropanol. The mixture was stirred until a homogenous solution was obtained. The mixture was cooled to 5° C. and HBr gas was bubbled in for 20 minutes with cooling. The mixture was placed in the refrigerator overnight. No solid material was formed. The mixture was then concentrated in vacuo to an oil and the oily residue was dissolved in acetone by heating to 45° C. (the solution was a 0.5 molar solution in acetone). The flask was scratched to initiate nucleation. The solution was cooled to 5° C. An off-white solid formed. The solid was collected, washed with cold acetone to give a crystalline material. The crystalline escitalopram hydrobromide was found by melting point, HPLC, and proton NMR to have a good purity. A sample of the material was exposed to air and it was found to be non-hygroscopic.

(B) Experiments with different solvents: These experiments were performed as follows: To a solution of the escitalopram free base (approx. 20% w/w) in dry iso-propanol was added dropwise 0.9-1.0 eq. of HBr (g) dissolved in dry iso-propanol. Precipitation of a solid normally occurred within 30 minutes. Where the precipitation was performed in a solvent other than iso-propanol, the resulting mixture was evaporated under reduced pressure and the appropriate solvent was added, evaporated again and the appropriate solvent given one more time to the mixture before final crystallisation.

Below is a table showing results from different solvents:

Precipitation of escitalopram hydrobromide from different solvents

| Solvent | Yield | Purity (HPLC) | Melting Point | |
|---|---|---|---|---|
| Toluene | 81% | 99.1% | 131° | C. |
| MTBE/IPA | 72% | 98.3% | 132° | C. |
| (200:55) | | | | |
| IPA | 67% | 99.4% | 133° | C. |
| MTBE | 93.4% | 99.2% | 131.6° | C. |
| THF | 54.5% | 99.95% | 133.9° | C. |
| Butanone | 30% | 100% | 133-134° | C. |
| n-Butanol | 67% | 99.9% | 133-134° | C. |
| iso-Butanol | 66% | 99.6% | 133-134° | C. |
| tert-Butanol/IPA (4:1) | 82% | 99.9% | 133-134° | C. |
| 2-Butanol | 85% | 100% | 133-134° | C. |

| | | | |
|---|---|---|---|
| MIBK | 75% | 100% | — |
| 2-methyl-THF | 84% | 100% | — |
| 1,4-Dioxane | 65% | 100% | — |
| Ether | 91% | 100% | — |
| EtOAc | 88% | 100% | — |

MTBE=methyl t-butyl ether; IPA=iso-propanol; MIBK=methyl iso-butyl ketone;

THF=tetrahydrofuran; EtOAc=ethyl acetate.

Other solvents, such as acetonitrile, methanol, ethanol and propylencarbonate, were tried, but gave no crystallisation:

### Example 2

Form I of escitalopram hydrobromide was characterized as follows.

1. X-Ray Powder Diffraction

X-ray powder diffraction analyses were carried out on a STOE Stadi P (available from STOE & CIE GmbH of Darmstadt, Germany) using Cu(Kα1) radiation. The parameters of the machine are shown below.
Diffractometer: STOE Stadi P
Radiation: Cu(Kα1), germanium monochromator, λ=1.540598
Position Sensitive Detector (PSD) covering 7°
Scan type: Stepscan, steps: 0.1°, 125-150 sec. pr. step
Range: 5-45°2θ
Sample measuring method: Transmission

The XRPD pattern for Form I prepared from an isopropanol and methyl t-butyl ether (MTBE) solution according to the procedure described in Example 1(B) is shown in FIG. 1.

2. Differential Scanning Calorimetry (DSC)

The melting point was determined using differential scanning calorimetry (DSC), using a TA instruments DSC 2920 (available from TA Instruments of New Castle, Del.) heating the sample 5°/min. The sample was placed in a covered pan. The DSC thermogram for Form I prepared from an isopropanol solution according to the procedure described in Example 1(B) is shown in FIG. 3. The Form I sample had an onset temperature at about 134.3° C. and a peak maximum at about 136.3° C. The enthalpy of fusion was about 67 J/g (27 kJ/mol).

3. Thermogravimetric Analysis (TGA)

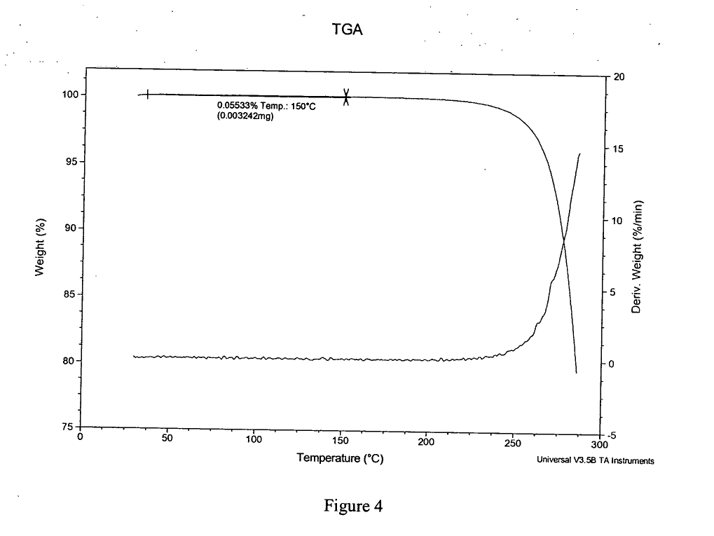

The thermogram for Form I prepared from an isopropanol solution according to the procedure described in Example 1(B) is shown in FIG. 4. The sample (1-5 mg) was heated 10°/min. on a TA Instruments TGA 2950 (available from TA Instruments of New Castle, Del.). No weight loss (<0.1%) was observed up to 150° C. Decomposition began at approximately 240° C.

4. Dynamic Vapor Sorption (DVS)

Dynamic Vapour Sorption (DVS) measurements were performed in order to determine whether Form I (prepared from an isopropanol solution according to the procedure described in Example 1(B)) is hygroscopic. Two cycles were made starting at 20% relative humidity and then equilibrating at the following relative humidity values: 20-30-40-50-60-70-80-90-95-90-80-70-60-50-40-30-20-10-0-10-20.

In the first run no water was absorbed until the relative humidity exceeded 70%. At 80% relative humidity, 14% was absorbed. 14% corresponds to about 3.6 mol-equivalent of water. After this the escitalopram hydrobromide was very hygroscopic and at 95% relative humidity almost 9 mol-equivalent of water was absorbed.

The isotherm plot is shown in FIG. 5.

5. X-Ray Single Crystal Structure Determination

The diffraction data for Form I were collected on a Nonius KappaCCD diffractometer having the parameters in the table below. The crystal was cooled down to 122±2 K in a stream of N₂ gas. All H-atoms appeared in a difference map. Subsequently the positions and isotropic displacement parameters were refined. The atom numbering used is shown in FIG. 2. The results are shown above.

Data Collection:

| Radiation | MoK_{α}, λ = 0.71073 Å |
|---|---|
| Absorption coefficient: | μ = 2.220 |
| Absorption correction | τₘᵢₙ = 0.40743, τₘₐₓ = 0.85800 |
| Temperature: | T = 122(2) K |
| Corrections: | Lorentz-polarization |
| hkl ranges: | h = −9 → 9 K = −15 → 15 l = −36 → 34 |
| No. of independent | 4927 |
| reflections: | |
| No. of reflections > 2sigma(I) | 4122 |

The crystallographic drawing in FIG. 2 was obtained using the program ORTEP.

All references, including patents, patent applications, publications, and procedures, cited throughout this application are incorporated herein by reference in their entireties.

1. Crystalline Form I of escitalopram hydrobromide.

2. A crystalline form of escitalopram hydrobromide that exhibits an x-ray powder diffraction pattern having characteristic peaks expressed in degrees 2θ at about 21.93±0.1 2θ.

3. The crystalline form of claim 2, further exhibiting characteristic peaks expressed in degrees 2θ at about 16.95, 18.59, 21.10, and 27.76±0.2 2θ.

4. A crystalline form of escitalopram hydrobromide that exhibits an x-ray powder diffraction pattern (using CuK_{α1} radiation) substantially the same as that shown in FIG. 1.

5. A crystalline form of escitalopram hydrobromide having a melting point onset as measured by differential scanning calorimetry at from about 131 to about 135° C.

6. A crystalline form of escitalopram hydrobromide that exhibits a single crystal X-ray crystallographic analysis at 122±2 K with crystal parameters that are approximately equal to the following:

| Parameter | Form I |
|---|---|
| Space group | Orthorhombic P2,2,2, |
| Cell Dimensions | |
| a(Å) | 6.5456 Å ± 8 Å |
| b(Å) | 11.0611 Å ± 6 Å |
| c(Å) | 25.795 Å ± 3 Å |
| Volume (Å³) | 1867.6 ± 3 Å³ |
| Z (molecules/unit cell) | 4 |
| Density | 1.442 g/cm³. |

7. A pharmaceutical composition comprising crystalline Form I of escitalopram hydrobromide and at least one pharmaceutically acceptable excipient.

8. The pharmaceutical composition of claim 7, wherein the pharmaceutical composition comprises at least about 90% by weight of Form I of escitalopram hydrobromide, based upon 100% total weight of escitalopram hydrobromide in the pharmaceutical composition.

9. A method for preparing crystalline escitalopram hydrobromide comprising the steps of:
(a) forming an anhydrous solution of escitalopram hydrobromide and at least one organic solvent; and
(b) precipitating crystalline escitalopram hydrobromide from the anhydrous solution.

10. The method of claim 9, wherein the organic solvent is iso-propanol.

11. The method of claim 9, wherein the organic solvent is selected from toluene, methyl t-butyl ether, a mixture of methyl t-butyl ether and isopropanol, tetrahydrofuran, butanone, n-butanol, iso-butanol, tert-butanol, a mixture of tert-butanol and isopropanol, 2-butanol, methyl iso-butyl ketone, 2-methyl-tetrahydrofuran, 1,4-dioxane, diethyl ether, ethyl acetate, acetone, and any combination of any of the foregoing.

12. The method of claim 9, wherein step (a) comprises:
(i) introducing hydrobromide gas into a solution of escitalopram free base and iso-propanol to form escitalopram hydrobromide;
(ii) concentrating the solution of step (i); and
(iii) dissolving the escitalopram hydrobromide from step (ii) in at least one organic solvent to form the anhydrous solution.

13. The method of claim 12, wherein the organic solvent in step (a)(iii) is acetone.

14. The method of claim 12, wherein the organic solvent in step (a)(iii) is selected from toluene, methyl t-butyl ether, a mixture of methyl t-butyl ether and isopropanol, tetrahydrofuran, butanone, n-butanol, iso-butanol, tert-butanol, a mixture of tert-butanol and isopropanol, 2-butanol, methyl iso-butyl ketone, 2-methyl-tetrahydrofuran, 1,4-dioxane, diethyl ether, ethyl acetate, acetone, and any combination of any of the foregoing.

15. The method of claim 9, wherein step (a) comprises adding a solution of hydrobromide and iso-propanol to a solution of escitalopram free base and iso-propanol to form the anhydrous solution.

16. The method of claim 9, wherein step (a) comprises:
(i) adding a solution of hydrobromide and iso-propanol to a solution of escitalopram free base and iso-propanol to form escitalopram hydrobromide;
(ii) concentrating the solution of step (i); and
(iii) dissolving the escitalopram hydrobromide from step (ii) in at least one organic solvent to form the anhydrous solution.

17. The method of claim 16, wherein the organic solvent is selected from toluene, methyl t-butyl ether, a mixture of methyl t-butyl ether and isopropanol, tetrahydrofuran, butanone, n-butanol, iso-butanol, tert-butanol, a mixture of tert-butanol and isopropanol, 2-butanol, methyl iso-butyl ketone, 2-methyl-tetrahydrofuran, 1,4-dioxane, diethyl ether, ethyl acetate, acetone, and any combination of any of the foregoing.

18. The method of claim 9, wherein the crystalline escitalopram hydrobromide comprises crystalline Form I of escitalopram hydrobromide.

19. A method for preparing crystalline escitalopram hydrobromide comprising the steps of:
(a) dissolving escitalopram free base in iso-propanol;
(b) adding aqueous hydrobromic acid;
(c) drying the solution of step (ii); and
(d) precipitating crystalline escitalopram hydrobromide from solution.

20. The method of claim 19, wherein step (c) comprises performing azeotropic distillation on the solution of step (ii).

21. The method of claim 19, wherein step (c) comprises adding a solid drying agent to the solution of step (ii).
